(19) United States
(12) Patent Application Publication
(10) Pub. No.: US 2023/0402160 A1
(43) Pub. Date: Dec. 14, 2023

(54) APPLICATION OF ARTIFICIAL INTELLIGENCE-BASED SOFTWARE FOR HEALTH INSURANCE PRIOR AUTHORIZATION APPROVAL IN MEDICAL DIAGNOSTICS AND INTERVENTIONS

(71) Applicant: Remote Health Ventures LLC, New York, NY (US)

(72) Inventors: Riley Williams, III, New York, NY (US); Benedict Nwachukwu, New York, NY (US)

(73) Assignee: Remote Health Ventures LLC, New York, NY (US)

(21) Appl. No.: 18/198,801

(22) Filed: May 17, 2023

Related U.S. Application Data
(60) Provisional application No. 63/342,765, filed on May 17, 2022.

(51) Int. Cl. G16H 40/20; G16H 30/40

(52) U.S. Cl. CPC G16H 40/20; G16H 30/40

(57) ABSTRACT

A method may receive a prior authorization approval request through a device connected to a network. The method may also obtain data from previous prior authorization decisions, documentation, socioeconomic variables, expert or peer review opinions, and imaging data, which may include medical imaging, histologic pathology, or serology data. Using one or more servers, the method may determine the extent of disease in a patient based on the imaging data. The method may further adjust prior authorization decision-making parameters by considering the expert or peer review opinions, previous decisions, documentation, socioeconomic variables, and imaging data. Finally, the method may generate a likelihood of prior authorization approval.

### CROSS REFERENCE TO RELATED APPLICATIONS

This application claims priority to U.S. Provisional Patent Application No. 63/342,765 filed on May 17, 2022. This application is also related to the following applications: U.S. Provisional Patent Application No. 62/704,160, filed on Apr. 24, 2020; U.S. Provisional Patent Application No. 63/015,256, filed on Apr. 24, 2020; U.S. Provisional Patent Application No. 62/706,139, filed on Aug. 3, 2020; U.S. Provisional Patent Application No. 62/706,142, filed on Aug. 3, 2020; U.S. patent application Ser. No. 17/239,939, filed on Apr. 26, 2021. Applicant incorporates by reference the disclosures of these applications.

### FIELD OF THE DISCLOSURE

The field of this invention addresses the present difficulties in simultaneously assimilating and analyzing multiple channels of clinical, social, and financial data to readily provide real-time feedback on the likelihood of receiving prior authorization approval.

### INTRODUCTION

The complex process of requesting and achieving prior authorization from health insurance payers prior to a medical diagnostic test or intervention presently represents a complex resource-intensive process for patients, healthcare providers, and insurers (i.e. payers). The input data involved in this process requires careful organization, analysis, and weighted consideration prior to authorizing the services and subsequent payment by the health insurance payer. Specifically, “prior authorization” involves the nature of the process by which insurers remotely weigh the cost and clinical effectiveness of a provider-ordered diagnostic test or medical intervention. Presently, this process causes delays in care, engenders confusion and miscommunication, and exacerbates the resources of all involved parties.

The payer prior authorization approval process seeks to make appropriate decisions in an expeditious manner but is limited by its capability to assimilate a multitude of data and possess clinical appropriateness knowhow in the absence of uniform practice guidelines.

Clinical and socioeconomic patient data, alongside the documented request of a healthcare provider, are aggregated and submitted to payers for internal analysis and evaluation. In this complex process, a combination of unverified internal parameters exist and are used to determine whether or not a healthcare service (i.e. diagnostic test or medical intervention) will be subsidized by the payer. In cases of discrepancy, more information, other tests, or third party adjudication may be requested by the payer from the provider. This process is resource- and time-intensive for all involved parties, from the patient to the provider to the payer. The complex nature of this decision-making process by the insurers requires consideration of many variables with various weights, as well as the opinion of a “peer” in certain situations. Historical and new data is prerequisite in advising on these prior authorization decisions. Regardless, these decisions require storage and management of tremendous disorganized, heterogenous data and necessitate automation to expedite decisions that impact clinical encounters and cost. Artificial intelligence-based analytic processes are capable of automated, efficient interpretation of data while assimilating aggregate historical and new input to arrive at rapid prior authorization decisions.

Accordingly, it is desirable to have methods and systems capable of efficiently provide an insight into the likelihood of prior authorization approval request being approved and/or a obtaining a swift decision thereof.

### SUMMARY

Aspects of the present disclosure relate to a method for assessing prior authorization approval, the method including: receive, via a device connected to a network via one or more servers, a prior authorization approval request; receive, via one or more servers, data from one or more previous prior authorization decisions, documentation, and socioeconomic variables, wherein at least a portion of the documentation is received from the prior authorization approval request; receive, via the one or more servers, data from one or more expert or peer review opinions; receive, via the one or more servers, imaging data, wherein the imaging data includes one or more of medical imaging, histologic pathology, or serology data; determine, via the one or more servers, an extent of disease of a patient based at least on the imaging data; adjust, via the one or more servers, one or more prior authorization decision-making parameters based on the one or more expert or peer review opinions, previous prior authorization decisions, documentation, socioeconomic variables, and imaging data; and generate, via the one or more servers, a likelihood of prior authorization approval.

Aspects of the present disclosure relate to a method, wherein the method further includes: generate, via the one or more servers, a recommendation for a course of clinical action if the prior authorization is denied.

Aspects of the present disclosure relate to a method, wherein the method further includes: generate, via the one or more servers, a prior authorization approval request decisions based one or more prior authorization decision-making parameters.

Aspects of the present disclosure relate to a method, wherein determining, via the one or more servers, the extent of disease of the patient is executed via one or more algorithms.

Aspects of the present disclosure relate to a method, wherein the one or more algorithms are machine learning algorithms, the machine learning algorithms include a computer-implemented method of determining the extent of disease of the patient including: receiving an input dataset including the imaging data; determining the extent of disease of the patient; and producing an output dataset including the extent of disease of the patient.

Aspects of the present disclosure relate to a method, wherein adjusting, via the one or more servers, the one or more prior authorization decision-making parameters, is executed via one or more algorithms.

Aspects of the present disclosure relate to a method, wherein the one or more algorithms are machine learning algorithms, the machine learning algorithms include a computer-implemented method of adjusting the one or more prior authorization decision-making parameters including: receiving an input dataset including: the one or more expert or peer review opinions, previous prior authorization decisions, documentation, socioeconomic variables, and imaging data; determining the one or more prior authorization decision-making parameters; and producing an output of adjusting the one or more prior authorization decision-making parameters.

Aspects of the present disclosure relate to a method, wherein generating, via the one or more servers, the likelihood of prior authorization approval, is executed via one or more algorithms.

Aspects of the present disclosure relate to a method, wherein the one or more algorithms are machine learning algorithms, the machine learning algorithms include a computer-implemented method of generating the likelihood of prior authorization approval including: receiving an input dataset including: the data from the one or more previous prior authorization decisions, documentation, and socioeconomic variables, the data from the one or more expert or peer review opinions, the imaging data, the extent of disease of the patient, and the one or more prior authorization decision-making parameters; determining the likelihood of prior authorization approval; and producing an output dataset including the likelihood of prior authorization approval.

Aspects of the present disclosure relate to a method, wherein the one or more prior authorization decision-making parameters include a weight, and wherein the weight is determined based on current and past expert or peer review opinions, previous prior authorization decisions, documentation, socioeconomic variables, and imaging data.

Aspects of the present disclosure relate to a system for assessing prior authorization approval, the system including one or more computer processors, and a memory having stored therein machine executable instructions, that when executed by the one or more processors, cause the system to: receive, via a device connected to a network via one or more servers, a prior authorization approval request; receive, via one or more servers, data from one or more previous prior authorization decisions, documentation, and socioeconomic variables, wherein at least a portion of the documentation is received from the prior authorization approval request; receive, via the one or more servers, data from one or more expert or peer review opinions; receive, via the one or more servers, imaging data, wherein the imaging data includes one or more of medical imaging, histologic pathology, or serology data; determine, via the one or more servers, an extent of disease of a patient based at least on the imaging data; adjust, via the one or more servers, one or more prior authorization decision-making parameters based on the one or more expert or peer review opinions, previous prior authorization decisions, documentation, socioeconomic variables, and imaging data; and generate, via the one or more servers, a likelihood of prior authorization approval.

Aspects of the present disclosure relate to a system, wherein the machine executable instructions, when executed by the one or more processors, further cause the system to: generate, via the one or more servers, a recommendation for a course of clinical action if the prior authorization is denied.

Aspects of the present disclosure relate to a system, wherein the machine executable instructions, when executed by the one or more processors, further cause the system to: generate, via the one or more servers, a prior authorization approval request decisions based one or more prior authorization decision-making parameters.

Aspects of the present disclosure relate to a system, wherein determining, via the one or more servers, the extent of disease of the patient is executed via one or more algorithms.

Aspects of the present disclosure relate to a system, wherein the one or more algorithms are machine learning algorithms, the machine learning algorithms include a computer-implemented method of determining the extent of disease of the patient including: receiving an input dataset including the imaging data; determining the extent of disease of the patient; and producing an output dataset including the extent of disease of the patient.

Aspects of the present disclosure relate to a system, wherein adjusting, via the one or more servers, the one or more prior authorization decision-making parameters, is executed via one or more algorithms.

Aspects of the present disclosure relate to a system, wherein the one or more algorithms are machine learning algorithms, the machine learning algorithms include a computer-implemented method of adjusting the one or more prior authorization decision-making parameters including: receiving an input dataset including: the one or more expert or peer review opinions, previous prior authorization decisions, documentation, socioeconomic variables, and imaging data; determining the one or more prior authorization decision-making parameters; and producing an output of adjusting the one or more prior authorization decision-making parameters.

Aspects of the present disclosure relate to a system, wherein generating, via the one or more servers, the likelihood of prior authorization approval, is executed via one or more algorithms.

Aspects of the present disclosure relate to a system, wherein the one or more algorithms are machine learning algorithms, the machine learning algorithms include a computer-implemented method of generating the likelihood of prior authorization approval including: receiving an input dataset including: the data from the one or more previous prior authorization decisions, documentation, and socioeconomic variables, the data from the one or more expert or peer review opinions, the imaging data, the extent of disease of the patient, and the one or more prior authorization decision-making parameters; determining the likelihood of prior authorization approval; and producing an output dataset including the likelihood of prior authorization approval.

Aspects of the present disclosure relate to a computer-readable storage medium having data stored therein representing software executable by a computer, the software having instructions to: receive, via a device connected to a network via one or more servers, a prior authorization approval request; receive, via one or more servers, data from one or more previous prior authorization decisions, documentation, and socioeconomic variables, wherein at least a portion of the documentation is received from the prior authorization approval request; receive, via the one or more servers, data from one or more expert or peer review opinions; receive, via the one or more servers, imaging data, wherein the imaging data includes one or more of medical imaging, histologic pathology, or serology data; determine, via the one or more servers, an extent of disease of a patient based at least on the imaging data; adjust, via the one or more servers, one or more prior authorization decision-making parameters based on the one or more expert or peer review opinions, previous prior authorization decisions, documentation, socioeconomic variables, and imaging data; and generate, via the one or more servers, a likelihood of prior authorization approval.

The features and advantages described in the specification are not all inclusive and, in particular, many additional features surrounding automated decision-making of health insurance prior authorization approval will be apparent to one of ordinary skill in the art in view of the specifications and claims.

Moreover, it should be noted that the language used in the specification has been principally selected for readability and instructional purposes, and may not have been selected to delineate or circumscribe the inventive subject matter.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

FIG. 1 illustrates a block diagram of a distributed computer system that can implement one or more aspects of the present disclosure.

FIG. 2 illustrates a block diagram of an electronic device that can implement one or more aspects of the present disclosure.

FIG. 3 illustrates an embodiment of the System according to one or more aspects of the present disclosure.

FIG. 4 illustrates a method for executing one or more aspects of the present disclosure.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

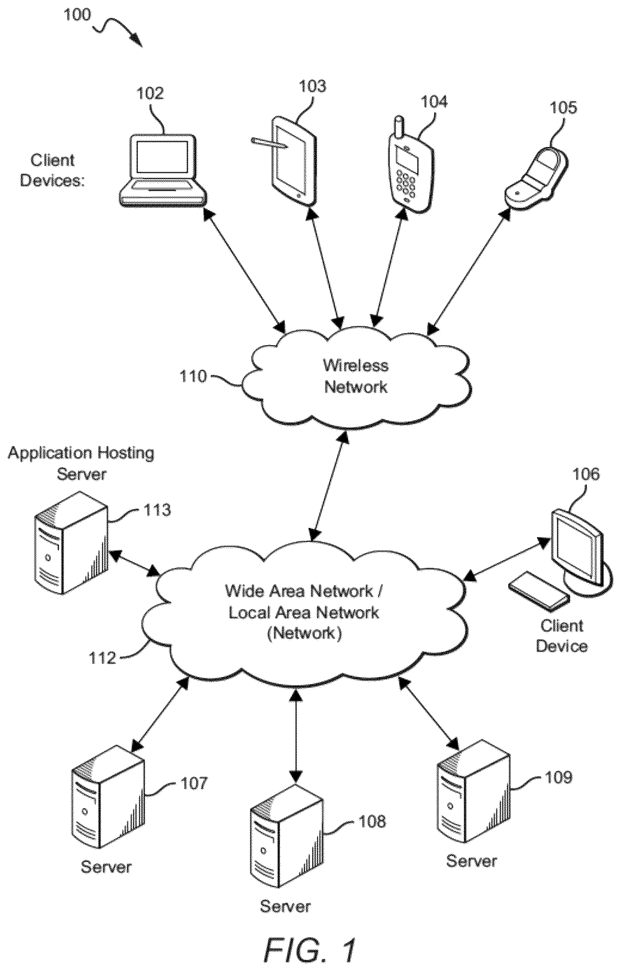

FIG. 1 illustrates components of one embodiment of an environment in which aspects of the present disclosure may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like. FIG. 1 also illustrates application hosting server 113.

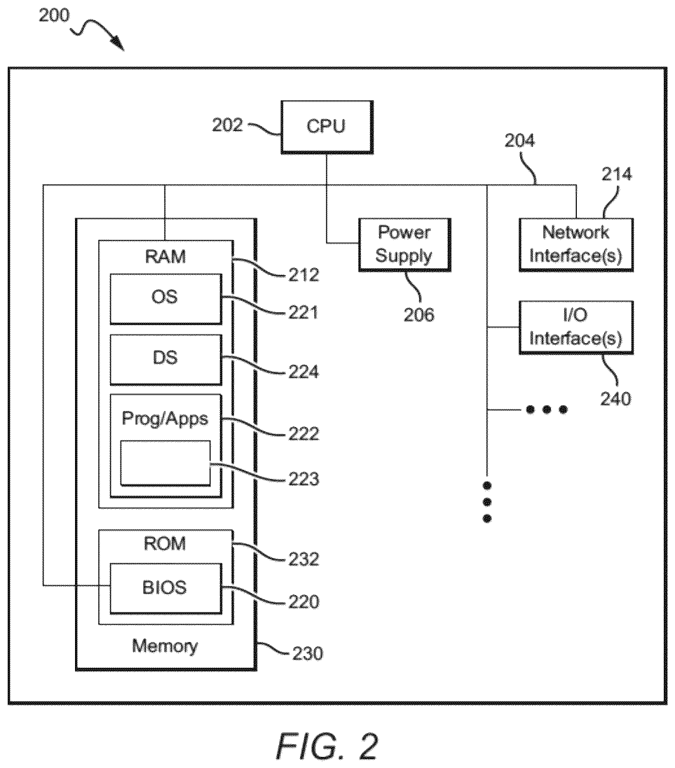

FIG. 2 illustrates a block diagram of an electronic device 200 that can implement one or more aspects of an apparatus, system and/or method for of the present disclosure (the “Engine”). Instances of the electronic device 200 may include servers, e.g., servers 107-109, and client devices, e.g., client devices 102-106. In general, the electronic device 200 can include a CPU/processor 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user's body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it may include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party's web site infrastructure, in whole or in part, on the third party's behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present disclosure include apparatuses, systems, and methods implementing the Engine. Embodiments of the present disclosure may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

Aspects of the present disclosure may utilize various Artificial Intelligence (AI) tools including Natural Language Processing (NLP).

NLP may facilitate the interaction between computers and human language. It may allow computers to understand, interpret, and generate human language, thereby processing and deriving potential meaning from text or speech data. NLP employs a combination of linguistic, statistical, and machine learning techniques that may assist in extracting information, classifying text, and generating potential responses.

The process of NLP may involve several steps. Initially, text data may be preprocessed, which may involve actions such as tokenization (breaking the text into individual words or tokens), the removal of stop words (such as common words like “and” or “the” that may carry less meaning), and stemming (which may reduce words to their root form). Subsequently, various techniques, as understood to those of skill in the art, may be employed to understand the structure and potential meaning of the text.

One component of NLP may be syntactic analysis or parsing, which may entail determining the grammatical structure of a sentence. This may aid in identifying relationships between words, such as subject-verb-object, and understanding the overall syntax of the sentence.

Semantic analysis is another facet of NLP that may seek to comprehend the meaning of words and phrases in context. It may involve tasks such as word sense disambiguation (identifying the correct meaning of a word with multiple meanings), named entity recognition (the identification and classification of named entities such as people, organizations, or locations), and sentiment analysis (determining the emotional tone of the text).

NLP may also encompass the use of statistical methods, including machine learning algorithms that may be trained on substantial datasets. These models may be utilized for diverse tasks such as text classification, language translation, question answering, and text generation. Neural networks, particularly deep learning models, have demonstrated notable success in NLP tasks, utilizing recurrent neural networks (RNNs) and transformer models, among others.

NLP may employ a combination of linguistic rules, statistical techniques, and machine learning algorithms to facilitate computer processing and potential understanding of human language.

The various Artificial Intelligence (AI) tools by aspects of the present disclosure may also include machine learning. Machine Learning may enable systems to learn from data and improve their performance without explicit programming. For example, machine learning may encompass various algorithms and techniques that may automatically analyze data, identify patterns, and make predictions or decisions.

Machine learning models may be trained using one or more algorithms that automatically analyze data, identify patterns, and make predictions or decisions. This iterative learning process may adjust model parameters based on input data, improving performance over time. Machine Learning may encompass techniques like supervised learning (learning from labeled examples), unsupervised learning (finding patterns in unlabeled data), and reinforcement learning (optimizing actions based on feedback).

Subsets of machine learning may include deep learning which may also be utilized by aspects of the present disclosure. Deep Learning may focus specifically on training deep neural networks with multiple layers to learn and represent complex patterns in data.

Deep Learning models, often referred to as deep neural networks, may leverage neural networks with multiple hidden layers to automatically extract hierarchical representations from data. These deep neural networks excel at tasks involving high-dimensional and complex data, such as images, audio, and natural language.

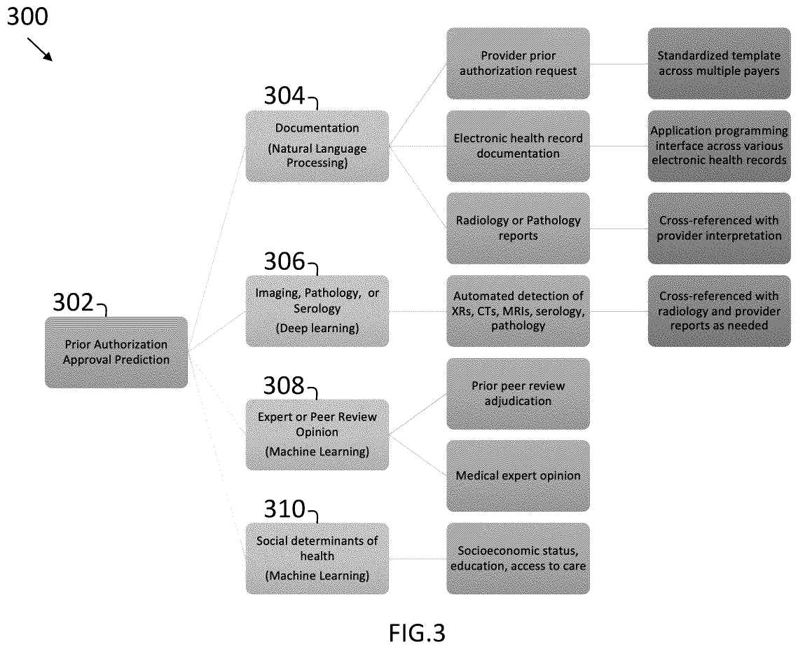

FIG. 3 illustrates an embodiment of the System according to one or more aspects of the present disclosure. The software application 300 may provide a prior authorization approval prediction 302 by applying various artificial-intelligence based processes in a unique organizational structure for a specific application and may be considered into four core data structures and potential embodiments: (1) Documentation 304; (2) Imaging 306; (3) Expert or Peer Review Opinion 308; and (4) Social Determinants of Health 310.

Aspects of the present disclosure relate to prior authorization approval process software 300 which may be initiated between devices 102-106 with displays, with zero or more sensors such as a camera, that may be any device 102-106 capable of accepting wireless data transfer, such as a smartphone, tablet, desktop, or a laptop. The display may be a visual screen that both the user and healthcare provider can read. The display may include, but is not limited to, an LED display, OLED display, AMOLED display, MicroLED display, LCD display, electronic ink (e-ink) display, plasma display, ELD display, and/or any other suitable display. The display may be mounted on other surfaces. In one embodiment, both devices 102-106 have computing power sufficient to run the software application.

Persons having skill in the art will realize that communication between devices 102-106 is not necessarily direct between the two devices 102-106 and could instead be indirect, via one or more intermediary devices 102-106 and/or networks 110/112 such as the Internet. The software application may interact with the devices 102-106 partaking in the telemedicine encounter through an API. The software application may be adapted to interact with a variety of APIs on multiple platforms.

In one embodiment, the software application 300 for prior authorization approval in medical diagnostics and interventions is initiated when a clinical order or authorization approval request is submitted by the provider to the health insurance management company or payer.

Documentation 304 may include the prior authorization approval request and related data, electronic health record documentation, and/or radiology or pathology reports as described herein. Because each payer has differing processes for submitting core features of the prior authorization approval request, basic data may be extracted and populated using NLP and may be used to fulfill the submission request for maximal interoperability and scalability. Such basic data may include, but is not limited to, patient information, healthcare provider information, insurance information, medical service or treatment details, supporting documentation, diagnosis and clinical information, and/or prescribing provider information.

Patient information may include information that is specific to the patient such as the patient's name, date of birth, gender, address, contact information, and insurance policy or ID number.

The healthcare provider information may include the name, address, and contact information of the healthcare provider submitting the request. It may also include the provider's National Provider Identifier (NPI) or other identification numbers.

The insurance information may include the name of the health insurance company, the policyholder's name (if different from the patient), the insurance group or plan number, and any relevant coverage details.

The medical service or treatment details may include a description of the requested medical service, treatment, or procedure. It may also include the CPT (Current Procedural Terminology) code or a detailed narrative explaining the procedure or treatment.

Supporting documentation depends on the nature of the prior authorization approval request, but may include medical records, test results, clinical notes, imaging reports, or any other relevant documents that provide justification for the requested service.

The diagnosis and clinical information may include the primary diagnosis or reason for the requested service or treatment. It may also include relevant medical history, previous treatments, and any other information that supports the medical necessity of the requested service.

If the prior authorization approval request is for medication, the prescribing provider information may include the prescribing provider's name, NPI, contact information, and their DEA (Drug Enforcement Administration) number if applicable.

Once the prior authorization approval request is submitted to the appropriate payer, this may fulfill the first component of Documentation 304. In another embodiment, Documentation 304 may include provider notes from the patient encounter that will similarly be processed for features including the history of present illness surrounding the disease (i.e., chronicity, impact, previously attempted modalities), the physical exam findings, the overall provider assessment, and future plan for the care of the patient. This data may be extracted from the electronic health record through an API or electronic transmission and analyzed using NLP.

In another embodiment, Documentation 304 may include additional supportive documentation. Such documentation may include the accompanying radiologist and pathologist reports, or the like, related to the disease and prior authorization approval request. This may similarly be extracted from the electronic health record through an API or electronic transmission and may be analyzed using NLP.

Turning to the imaging data 306. In one embodiment, medical imaging (i.e., radiographic imaging), histologic pathology, and/or serology data (i.e. laboratory specimens and values) may be extracted, analyzed, and processed to aid in the decision-making process for prior authorization approval. Extracted data may be preprocessed (i.e., cropped, converted, resized) in terms of pixels, trends, or other signal from an image, video, quantitative value, or the like. Such functions may be executed via deep learning algorithms.

For this extracted and preprocessed data, various algorithms may be applied such as a Convolutional Neural Network, other Artificial Neural Networks (ANN), k-Nearest Neighbor (kNN), Naïve Bayes, Support Vector Machines (SVM), and Decision Trees to aid in clinical diagnosis or classification. Heatmaps, class activation maps, or Shapley Additive Explanation summary aggregate plots may be subsequently generated for feedback in one embodiment.

The above algorithms may be used to detect and log specific data relating to X-rays, computerized tomography (CT) scans, Magnetic Resonance Imaging (MRI), serology, and/or pathology from the medical imaging, histologic pathology, and/or serology data. From such data, the software application 300 may determine the extent of a patient's disease. This may be achieved from the inputs received from the software application 300.

In one embodiment, Expert or Peer review opinion 308 may be considered via prior peer review decisions/adjudications or newly introduced medical expert opinion. Once the opinion of a singular or panel of Experts or Peers is solicited, a classification and interpretation algorithm may be applied, such as a machine learning algorithm, a pattern recognition algorithm, a template matching algorithm, a statistical inference algorithm, and/or an artificial intelligence algorithm that operates based on a learning model. Examples of such algorithms include, but are not limited to, kNN, Naïve Bayes, SVM, ANN, and Decision Trees. If the documentation of the Expert or Peer Review is only available as written documentation, NLP may be applied for scanning and data extraction.

In an embodiment, socioeconomic variables/social determinants of health 310 may be subsequently aggregated and similarly analyzed using kNN, Naïve Bayes, SVM, ANN, and Decision Trees from public or private databases that provide further definition to the patient, provider, or payer. Such data may include, for example, income, race; employment status; number of dependents; level of education; access to healthcare; any outstanding claims related to the patient, payer, or provider; or any other suitable socioeconomic/social factors known to those of skill in the art.

In one embodiment, some or all aforementioned embodiments of the prior authorization approval process executed by the software application 300 including, but not limited to, Documentation 304, Imaging 306, Expert or Peer Review Opinion 308, and/or Social Determinants of Health 310, may be processed simultaneously or in sequence using artificial intelligence-based decision supportive processes and parameters.

The software application 300 may, using the processes described herein, distill the input information (such as the documentation 304, imaging data 306, expert or peer opinion(s) 308, and/or social determinants of health 310) into one or more of (1) disorganized and infinite documentation phrases amenable to search and natural language processing techniques; (2) representations, screen captures, or complete medical imaging such as x-rays, CT scans, or Mills that may have image splicing and processing techniques applied and may be amenable to rapid automated interpretation by the software application 300 or expert opinion assessment; and/or (3) binary data representing opinions on whether or not the clinical decision warrants prior authorization approval.

These parameters from the aforementioned extracted data may carry unspecified weights. In an embodiment, the weights may be specified by a user. As a non-limiting example, the user may specify the following weights:
Documentation 304: 20%
Imaging data 306: 20%
Expert or Peer Review Opinion 308: 40%
Social Determinants of Health 310: 20%

In another embodiment, the weights may be set by the software application 300 based upon current and past input data including documentation 304, imaging data 306, Expert or Peer Review Opinion 308, and/or Social Determinants of Health 310.

The parameters from the aforementioned extracted data may be combined with preexisting verdicts surrounding past prior authorization decisions. For example, if the patient has a prior authorization history containing a majority of denials, the algorithms disclosed herein may decrease the likelihood of a prior authorization approval accordingly. Similarly, these parameters surrounding prior authorization approvals may be amenable to new evidence, medical expertise opinion, or modification with new data. In other words, the software application 300 may analyze and weigh the aforementioned objective and subjective data from insurer, patient, provider, and/or arbitration panel users through standard artificial intelligence-based processes to arrive at a recommendation for prior authorization approval while retaining the capacity to iteratively improve.

The method by which these processes are simultaneously weighed by the software application 300 may be performed using any myriad of artificial intelligence-based processes, networks, or algorithms such as kNN, Naïve Bayes, SVM, ANN, or Decision Tree. The output of such an embodiment may provide a prediction, recommendation, or likelihood in the form of a binary response or quantitative likelihood of receiving or recommending prior authorization approval from a specific health insurance payer. In the event a negative recommendation (i.e. prior authorization approval request denial or low likelihood of receiving approval) occurs, one embodiment may provide a recommendation to fulfill missing elements that may result in a positive recommendation (i.e. prior authorization approval request approval or high likelihood of receiving approval), such as additional documentation, further imaging, continued observation, or other clinical recommendations.

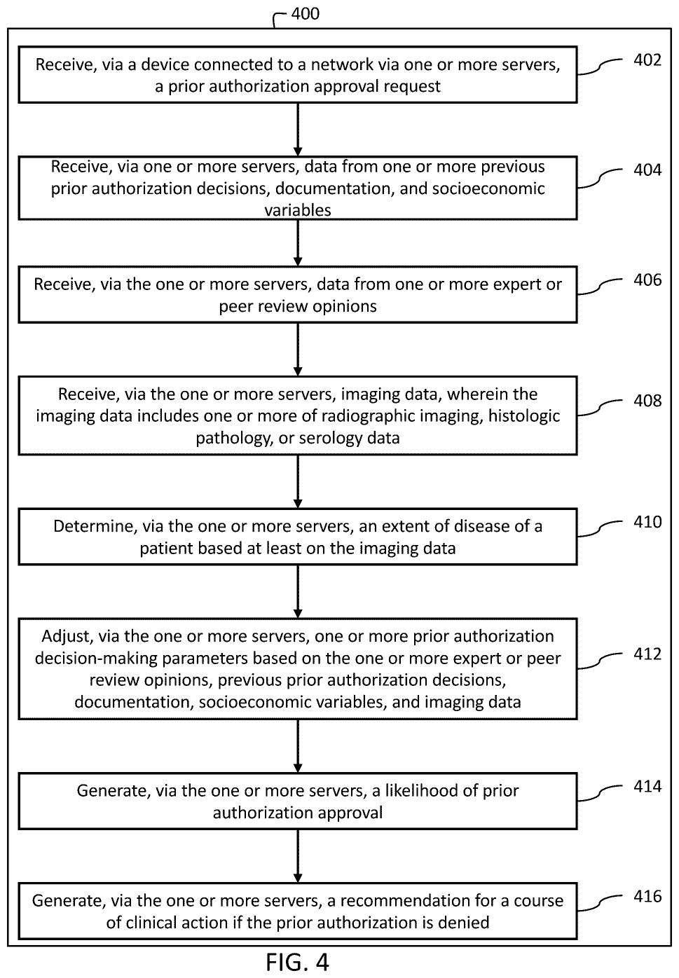

One or more aspects of the present disclosure may be executed via the method 400 illustrated in FIG. 4. The method 400 may enable a system that, in one or more embodiments, (1) aggregates and organizes data from previous prior authorization decisions, clinical data (i.e. note documentation, radiology reports), and socioeconomic variables (i.e. age, weight); (2) aggregates and organizes data from expert or peer review adjudication of prior authorization; (3) evaluates the extent of disease based on imaging with or without radiologist reports; (4) adjusts prior authorization decision-making parameters based on expert adjudication, updated clinical practice guidelines, or new evidence; (5) provides a recommendation of whether prior authorization approval from the insurer is likely; and (6) provides a recommendation for a course of clinical action where appropriate if prior authorization approval is denied. As the insurer or provider inputs more data into the device 102-106, or servers 107-109 that host the embodiment, iterative improvement may occur when recommended prior authorization approvals are compared to final prior authorization verdicts to further optimize the automated system using artificial intelligence-based processes.

Starting with step 402, the software application 300 may receive a prior authorization approval request. The prior authorization request may be received via a device 102-106 connected to a network 110/112 via one or more servers 107-109. The prior authorization approval request may include at least the basic data described herein including patient information, healthcare provider information, insurance information, medical service or treatment details, supporting documentation, diagnosis and clinical information, and/or prescribing provider information.

At step 404, the software application 300 may receive data from one or more previous prior authorization decisions, documentation, and/or socioeconomic variables. In an embodiment, at least a portion of the documentation is obtained from the prior authorization approval request. The socioeconomic variables may include social determinants of health 310. The one or more previous prior authorization decisions, documentation, and/or socioeconomic variables may be stored on one or more servers 107-109. To convert the prior authorization decisions, documentation, socioeconomic variables, various AI algorithms may be used as described herein.

Turning to step 406, the software application 300 may receive data from one or more expert or peer review opinions 308.

Next, at step 408, the software application 300 may receive imaging data 306. The imaging data 306 may include one or more of medical imaging, histologic pathology, or serology data.

At step 410, the software application 300 may determine an extent of disease of a patient. Such an extent of disease may be based at least on the imaging data 306. However, additional factors may be used, such as the documentation 304, Expert or Peer Review Opinion 308, and/or social determinants of health 310.

At step 412, the software application 300 may adjust one or more prior authorization decision-making parameters based on the one or more expert or peer review opinions 308, previous prior authorization decisions, documentation 304, socioeconomic variables/social determinants of health 310, and/or imaging data 306.

Turning to step 414, the software application 300 may generate a likelihood of prior authorization approval. Such a likelihood may be based upon the adjusted one or more prior authorization decision-making parameters.

At step 416, the software application 300 may generate a recommendation for a course of clinical action if the prior authorization is denied. In an embodiment, step 416 may not execute if the likelihood of prior authorization approval meets and/or exceeds a certain threshold. For example, the software application 300 may not execute step 416 if the likelihood of prior authorization approval exceeds 50%.

The software application 300 may generate prior authorization approval decisions based on inputs including, but not limited to, the documentation 304, imaging data 306, expert or peer opinion(s) 308, and/or social determinants of health 310 using the same processes described herein for determining the likelihood of prior authorization approval. For example, the software application 300 may be configured to approve a prior authorization approval request if the likelihood of prior authorization approval exceeds 60%. However, any suitable threshold may be configured.

In an embodiment, the software application 300 generates prior authorization approval decisions based on input from the provider's authorization approval request, documentation 304 and variables from the electronic medical record (such as imaging data 306), socioeconomic variables/social determinants of health 310 generated from the patient's submitted documentation to the insurer and prior claims. Data concerning previous prior authorization decisions may also be used. Such data may include the number of previous prior authorization requests submitted for the same issue, and expert or peer adjudication from previous, present, or future expert panels.

The software application 300 may generate metrics in real time to either the insurer, patient, arbiter, or healthcare provider to provide feedback in the form of a report that may be visual, audio, or written and potentially transmitted electronically. In an embodiment, the software application 300 is continuously iterating upon the multiple sources and formats of data, previous and current recommendations, and final prior authorization decisions across the various personnel users with respect to the requested intervention (i.e. elective hip replacement, shoulder MRI) necessitating prior authorization.

Reference in the specification to “one embodiment” or to “an embodiment” means that a particular feature, structure, or characteristic described in connection with the embodiment is included in at least one embodiment. The appearances of the phrase “in one embodiment” or “an embodiment” in various places in the specification are not necessarily all referring to the same embodiment.

Some portions of the detailed description are presented in terms of algorithms and symbolic representations of operations on data bits within a computer memory. These algorithmic descriptions and representations are the means used by those skilled in the art. An algorithm is here, and generally, conceived to be a self-consistent sequence of steps (instructions) leading to a desired result. The steps are those requiring physical manipulations of physical quantities. Usually, though not necessarily, these quantities take the form of electrical, magnetic, visual, auditory or optical signals capable of being stored, transferred, combined, compared and otherwise manipulated. It is convenient at times, principally for reasons of common usage, to refer to these signals as bits, values, pixels, elements, symbols, characters, terms, numbers, or the like. Furthermore, it is also convenient at times, to refer to certain arrangement of steps requiring physical manipulations or transformation of physical quantities or representations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or “determining” or the life, refer to the action and processes of a computer system, or similar electronic computing device (such as a specific computing machine), hat manipulates and transforms data represented as physical (electronic) quantities within the computing system memories or registers or other such information storage, transmission or display devices.

Certain aspects of the embodiments include process steps and instructions herein in the form of an algorithm. It should be noted that the process steps and instructions of the embodiments can be embodied in software, firmware or hardware, and when embodied in software, firmware or hardware, and when embodied in software could be downloaded to resided on and be operated from different platforms used by a variety of operating systems. The embodiments can also be in a computer program product, which can be executed on a computing system.

The embodiments also relate to an apparatus for performing the operations herein. This apparatus may be specially constructed for the purposes, e.g. a specific computer, or it may comprise a general purpose computer selectively activated or reconfigured by a computer program stored in the computer. Such a computer program may be stored in a computer readable storage medium, such as but is not limited to, any type of disk including floppy disks, optical disks, CD-ROMs, read-only memories (ROMs), random access memories (RAMs), EPROMs, EEPROMs, magnetic or optical cards, application specific integrated circuits (ASICs), or any type of media suitable for storing electronic instructions, and each coupled to a computer system bus. Memory can include any of the above and/or other devices that can store information/data/programs and can be transient or non-transient medium, where a non-transient or non-transitory medium can include memory/storage that stores information for more than a minimal duration. Furthermore, the computers referred to in the specifications may include a single processor or may be architectures employing multiple processor designs for increased computing capability.

The algorithms and displays presented herein are not inherently related to any particular computer or other apparatus. Various general-purpose systems may also be used with programs in accordance with the teachings herein, or it may prove convenient to construct more specialized apparatus to perform the method steps. The structure for a variety of these systems will appear from the description herein. In addition, the embodiments are not described with reference to any particular programming language. It will be appreciated that a variety of programming languages may be used to implement the teachings of the embodiments as described herein, and any references herein to specific languages are provided for disclosure of enablement and best mode. While particular embodiments and applications have been illustrated and described herein, it is to be understood that the embodiments are not limited to the precise construction and components disclosed herein and that various modifications, changes, and variations may be made in the arrangement, operation, and details of the methods and apparatuses of the embodiments without departing from the spirit and scope of the embodiments as defined in the appended claims.

Other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A method for assessing prior authorization approval, the method comprising:
receive, via a device connected to a network via one or more servers, a prior authorization approval request;
receive, via one or more servers, data from one or more previous prior authorization decisions, documentation, and socioeconomic variables,
wherein at least a portion of the documentation is received from the prior authorization approval request;
receive, via the one or more servers, data from one or more expert or peer review opinions;
receive, via the one or more servers, imaging data,
wherein the imaging data includes one or more of medical imaging, histologic pathology, or serology data;
determine, via the one or more servers, an extent of disease of a patient based at least on the imaging data;
adjust, via the one or more servers, one or more prior authorization decision-making parameters based on the one or more expert or peer review opinions, previous prior authorization decisions, documentation, socioeconomic variables, and imaging data; and
generate, via the one or more servers, a likelihood of prior authorization approval.

2. The method of claim 1, wherein the method further includes:
generate, via the one or more servers, a recommendation for a course of clinical action if the prior authorization is denied.

3. The method of claim 1, wherein the method further includes:
generate, via the one or more servers, a prior authorization approval request decisions based one or more prior authorization decision-making parameters.

4. The method of claim 1, wherein determining, via the one or more servers, the extent of disease of the patient is executed via one or more algorithms.

5. The method of claim 4, wherein the one or more algorithms are machine learning algorithms, the machine learning algorithms include a computer-implemented method of determining the extent of disease of the patient including:
receiving an input dataset comprising the imaging data;
determining the extent of disease of the patient; and
producing an output dataset including the extent of disease of the patient.

6. The method of claim 1, wherein adjusting, via the one or more servers, the one or more prior authorization decision-making parameters, is executed via one or more algorithms.

7. The method of claim 6, wherein the one or more algorithms are machine learning algorithms, the machine learning algorithms include a computer-implemented method of adjusting the one or more prior authorization decision-making parameters including:
receiving an input dataset comprising:
the one or more expert or peer review opinions, previous prior authorization decisions, documentation, socioeconomic variables, and imaging data;
determining the one or more prior authorization decision-making parameters; and
producing an output of adjusting the one or more prior authorization decision-making parameters.

8. The method of claim 1, wherein generating, via the one or more servers, the likelihood of prior authorization approval, is executed via one or more algorithms.

9. The method of claim 8, wherein the one or more algorithms are machine learning algorithms, the machine learning algorithms include a computer-implemented method of generating the likelihood of prior authorization approval including:
receiving an input dataset comprising:
the data from the one or more previous prior authorization decisions, documentation, and socioeconomic variables,
the data from the one or more expert or peer review opinions,
the imaging data,
the extent of disease of the patient, and
the one or more prior authorization decision-making parameters;
determining the likelihood of prior authorization approval; and
producing an output dataset comprising the likelihood of prior authorization approval.

10. The method of claim 1, wherein the one or more prior authorization decision-making parameters include a weight, and wherein the weight is determined based on current and past expert or peer review opinions, previous prior authorization decisions, documentation, socioeconomic variables, and imaging data.

11. A system for assessing prior authorization approval, the system comprising one or more computer processors, and a memory having stored therein machine executable instructions, that when executed by the one or more processors, cause the system to:
receive, via a device connected to a network via one or more servers, a prior authorization approval request;
receive, via one or more servers, data from one or more previous prior authorization decisions, documentation, and socioeconomic variables,
wherein at least a portion of the documentation is received from the prior authorization approval request;
receive, via the one or more servers, data from one or more expert or peer review opinions;
receive, via the one or more servers, imaging data,
wherein the imaging data includes one or more of medical imaging, histologic pathology, or serology data;
determine, via the one or more servers, an extent of disease of a patient based at least on the imaging data;
adjust, via the one or more servers, one or more prior authorization decision-making parameters based on the one or more expert or peer review opinions, previous prior authorization decisions, documentation, socioeconomic variables, and imaging data; and
generate, via the one or more servers, a likelihood of prior authorization approval.

12. The system of claim 11, wherein the machine executable instructions, when executed by the one or more processors, further cause the system to:
generate, via the one or more servers, a recommendation for a course of clinical action if the prior authorization is denied.

13. The system of claim 11, wherein the machine executable instructions, when executed by the one or more processors, further cause the system to:
generate, via the one or more servers, a prior authorization approval request decisions based one or more prior authorization decision-making parameters.

14. The system of claim 11, wherein determining, via the one or more servers, the extent of disease of the patient is executed via one or more algorithms.

15. The system of claim 14, wherein the one or more algorithms are machine learning algorithms, the machine learning algorithms include a computer-implemented method of determining the extent of disease of the patient including:
receiving an input dataset comprising the imaging data;
determining the extent of disease of the patient; and
producing an output dataset including the extent of disease of the patient.

16. The system of claim 11, wherein adjusting, via the one or more servers, the one or more prior authorization decision-making parameters, is executed via one or more algorithms.

17. The system of claim 16, wherein the one or more algorithms are machine learning algorithms, the machine learning algorithms include a computer-implemented method of adjusting the one or more prior authorization decision-making parameters including:
receiving an input dataset comprising:
the one or more expert or peer review opinions, previous prior authorization decisions, documentation, socioeconomic variables, and imaging data;
determining the one or more prior authorization decision-making parameters; and
producing an output of adjusting the one or more prior authorization decision-making parameters.

18. The system of claim 11, wherein generating, via the one or more servers, the likelihood of prior authorization approval, is executed via one or more algorithms.

19. The system of claim 18, wherein the one or more algorithms are machine learning algorithms, the machine learning algorithms include a computer-implemented method of generating the likelihood of prior authorization approval including:
receiving an input dataset comprising:
the data from the one or more previous prior authorization decisions, documentation, and socioeconomic variables,
the data from the one or more expert or peer review opinions,
the imaging data,
the extent of disease of the patient, and
the one or more prior authorization decision-making parameters;
determining the likelihood of prior authorization approval; and
producing an output dataset comprising the likelihood of prior authorization approval.

20. A computer-readable storage medium having data stored therein representing software executable by a computer, the software having instructions to:
receive, via a device connected to a network via one or more servers, a prior authorization approval request;
receive, via one or more servers, data from one or more previous prior authorization decisions, documentation, and socioeconomic variables,
wherein at least a portion of the documentation is received from the prior authorization approval request;
receive, via the one or more servers, data from one or more expert or peer review opinions;
receive, via the one or more servers, imaging data,
wherein the imaging data includes one or more of medical imaging, histologic pathology, or serology data;
determine, via the one or more servers, an extent of disease of a patient based at least on the imaging data;
adjust, via the one or more servers, one or more prior authorization decision-making parameters based on the one or more expert or peer review opinions, previous prior authorization decisions, documentation, socioeconomic variables, and imaging data; and
generate, via the one or more servers, a likelihood of prior authorization approval.
